(19) United States
(12) Patent Application Publication
(10) Pub. No.: US 2024/0239748 A1
(43) Pub. Date: Jul. 18, 2024

(54) NOVEL FORMS OF ENTINOSTAT

(71) Applicant: Macfarlan Smith Limited, Wayne, PA (US)

(72) Inventors: Thierry BONNAUD, Cambridge (GB); Zoe PRENTICE, Cambridge (GB)

(21) Appl. No.: 18/560,076

(22) PCT Filed: May 10, 2022
(86) PCT No.: PCT/EP2022/062611

§ 371 Date: Nov. 9, 2023

Related U.S. Application Data
(60) Provisional application No. 63/201,704, filed on May 10, 2021.

(51) Int. Cl. C07D 213/55; A61K 31/4406

(52) U.S. Cl. CPC C07D 213/55; A61K 31/4406

(57) ABSTRACT

The present disclosure relates to various crystalline forms of entinostat and to processes for their preparation, particularly Form A of entinostat compound with maleic acid, Form A of entinostat compound with succinic acid, Form B of entinostat compound with succinic acid, and Form C of entinostat compound with succinic acid. The present disclosure also relates to pharmaceutical compositions comprising any of these forms of entinostat and to use of these forms in preparing a medicament or in treating a disease.

### FIELD OF THE DISCLOSURE

The present disclosure relates to various crystalline forms of entinostat and to processes for their preparation. The present disclosure also relates to pharmaceutical compositions comprising any of these forms of entinostat and to use of these forms in preparing a medicament or in treating a disease.

### BACKGROUND OF THE DISCLOSURE

Entinostat, N-(2-aminophenyl)-4-[N-(pyridine-3-yl)-methoxycarbonylamino-methyl]-benzamide, is a selective, oral, once-weekly, class I HDAC inhibitor that has been studied in multiple solid tumors in combination with hormone therapy and several approved PD-1/PD-L1 antagonists. Entinostat has the following structure:

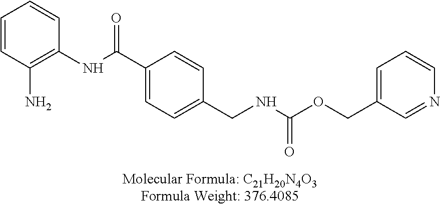 [SMILES: Nc1ccccc1NC(=O)c1ccc(CNC(=O)OCc2cccnc2)cc1]

U.S. Pat. No. 7,973,166 and RE45,499 disclose various polymorphs of entinostat, including Form A, Form B, Form C, and an amorphous form.

WO2017/081278 discloses additional polymorphs of entinostat, specifically Form D and Form E, and a process for preparing Form A. WO2017/216761 discloses an additional crystalline form, a stable amorphous form of entinostat, an amorphous solid dispersion of entinostat, and processes for their preparation.

U.S. Patent Application Publication No. 2020/0270213 discloses various co-crystals or salts of entinostat, including fumaric acid, succinic acid, maleic acid, and adipic acid co-crystals or salts of entinostat. The entinostat and maleic acid compound is described as having X-ray powder diffraction peaks measured using Cu-Ka radiation at 2θ values of 9.8°±0.2°, 15.9°±0.2°, 19.7°±0.2°, 23.2°±0.2°, 14.1°±0.2°, 14.8°±0.2°, 17.7°±0.2°, 18.9°±0.2°, 14.5°±0.2°, 21.4°±0.2°, 23.8°±0.2° and 24.8°±0.2°. The entinostat and succinic acid compound is described as having X-ray powder diffraction peaks measured using Cu-Ka radiation at 20 values of 3.7°±0.2°, 7.3°±0.2°, 9.2°±0.2°, 9.5°±0.2°, 16.6°±0.2°, 17.2°±0.2°, 17.8°±0.2°, 18.5°±0.2°, 19.0°±0.2°, 20.4°±0.2°, 20.8°±0.2° and 24.6°±0.2°.

None of these references disclose an entinostat co-crystal or salt formed with maleic acid having peaks at 18.6° and 18.1°±0.2° 20. Further, none of these references disclose an entinostat co-crystal or salt formed with succinic acid having any of the following: peaks at 18.7° and 18.3°±0.2° 2θ; two or more peaks selected from 21.5°, 26.5°, 23.9°, and 21.7°±0.2° 2θ; or two or more peaks selected from 4.0°, 8.0°, 15.7°, and 19.0°±0.2° 2θ.

### SUMMARY OF THE DISCLOSURE

The present invention is directed to various crystalline forms of entinostat, namely, an entinostat co-crystal or salt formed with maleic acid, referred to as Form A of entinostat compound with maleic acid, and three different polymorphs of an entinostat co-crystal or salt formed with succinic acid, namely, Form A of entinostat compound with succinic acid, Form B of entinostat compound with succinic acid, and Form C of entinostat compound with succinic acid. The present invention is further directed to processes for the preparation of these forms. The present invention also is directed to pharmaceutical compositions comprising one or more of these forms and to their use in preparing a medicament or in treating a disease.

### BRIEF DESCRIPTION OF THE DRAWINGS

FIG. 1 provides a representative XRPD pattern of Form A of entinostat compound with maleic acid.

FIG. 2 provides a representative 1H NMR of Form A of entinostat compound with maleic acid.

FIG. 3 provides a representative overlay of a TGA plot and DSC plot of Form A of entinostat compound with maleic acid.

FIG. 4 provides a representative XRPD pattern of Form A of entinostat compound with succinic acid.

FIG. 5 provides a representative 1H NMR of Form A of entinostat compound with succinic acid.

FIG. 6 provides a representative overlay of a TGA plot and DSC plot of Form A of entinostat compound with succinic acid.

FIG. 7 provides a representative GVS kinetic plot of Form A of entinostat compound with succinic acid.

FIG. 8 provides a representative GVS isotherm plot of Form A of entinostat compound with succinic acid.

FIG. 9 provides a representative XRPD pattern of Form B of entinostat compound with succinic acid.

FIG. 10 provides a representative 1H NMR of Form B of entinostat compound with succinic acid.

FIG. 11 provides a representative overlay of a TGA plot and DSC plot of Form B of entinostat compound with succinic acid.

FIG. 12 provides a representative XRPD pattern of Form C of entinostat compound with succinic acid.

FIG. 13 provides a representative 1H NMR of Form C of entinostat compound with succinic acid.

FIG. 14 provides a representative overlay of a TGA plot and DSC plot of Form C of entinostat compound with succinic acid.

### DETAILED DESCRIPTION OF THE DISCLOSURE

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles described herein may be applied to other examples and applications without departing from the spirit and scope of the various embodiments. Therefore, the various embodiments are not intended to be limited to the examples described herein and shown but are to be accorded the scope consistent with the claims.

### Terms/Definitions

As used herein and unless otherwise specified, the terms “about” and “approximately,” when used in connection with a numeric value or a range of values which is provided to characterize a particular solid form, e.g., a specific temperature or temperature range, such as, e.g., that describing a DSC or TGA thermal event, including, e.g., melting, dehydration, desolvation or glass transition events; a mass change, such as, e.g., a mass change as a function of temperature or humidity; a solvent or water content, in terms of, e.g., mass or a percentage; or a peak position, such as, e.g., in analysis by IR or Raman spectroscopy or XRPD; indicate that the value or range of values may deviate to an extent deemed reasonable to one of ordinary skill in the art while still describing the particular solid form.

As used herein and unless otherwise specified, the term “pharmaceutical composition” is intended to encompass a pharmaceutically effective amount of one or more of Form A of entinostat compound with maleic acid, Form A of entinostat compound with succinic acid, Form B of entinostat compound with succinic acid, and Form C of entinostat compound with succinic acid, and a pharmaceutically acceptable excipient. As used herein, the term “pharmaceutical compositions” includes pharmaceutical compositions such as tablets, pills, powders, liquids, suspensions, emulsions, granules, capsules, suppositories, or injection preparations.

As used herein and unless otherwise specified, the term “crystalline” and related terms used herein, when used to describe a compound, substance, modification, material, component or product, unless otherwise specified, mean that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, Md. (2005); The United States Pharmacopeia, 23rd ed., 1843-1844 (1995).

As used herein and unless otherwise specified, “co-crystal” and “co-crystal systems” refer to solid materials composed of two or more different components that are solid at room temperature and in particular stoichiometric ratios which interact through non-covalent interactions which can be designed utilizing supramolecular synthon approach. The co-crystal, in which at least one of the components is entinostat and the coformer is a second pharmaceutically acceptable compound, is called a pharmaceutical entinostat co-crystal with the coformer.

As used herein and unless otherwise specified, “salt” refers to a substance produced by the reaction of an acid with a base. A salt consists of the positive ion (cation) of a base and the negative ion (anion) of an acid. In this disclosure, “salt” refers to compounds formed between entinostat and maleic acid and to compounds formed between entinostat and succinic acid having the properties identified herein.

As used herein and unless otherwise specified, the term “excipient” refers to a pharmaceutically acceptable organic or inorganic carrier substance. Excipients may be natural or synthetic substances formulated alongside the active ingredient of a medication, included for the purpose of bulking-up formulations that contain potent active ingredients (thus often referred to as “bulking agents,” “fillers,” or “diluents”), or to confer a therapeutic enhancement on the active ingredient in the final dosage form, such as facilitating drug absorption or solubility. Excipients can also be useful in the manufacturing process, to aid in the handling of the active substance, such as by facilitating powder flowability or non-stick properties, in addition to aiding in vitro stability such as prevention of denaturation over the expected shelf life.

As used herein and unless otherwise specified, the term “patient” refers to an animal, preferably a mammal, most preferably a human, who has been the object of treatment, observation, or experiment. Preferably, the patient has experienced and/or exhibited at least one symptom of the disease or disorder to be treated and/or prevented. Further, a patient may not have exhibited any symptoms of the disorder, disease, or condition to be treated and/or prevented, but has been deemed by a physician, clinician, or other medical professional to be at risk for developing said disorder, disease, or condition.

As used herein and unless otherwise specified, the terms “polymorph,” “polymorphic form” or related term herein, refer to a crystalline form of an API (active pharmaceutical ingredient) free base or salt thereof that can exist in two or more forms, as a result of different arrangements or conformations of the molecule, ions of the salt, or addition and arrangement of solvents or coformers within the crystalline lattice of the crystal form.

As used herein and unless otherwise specified, the terms “substantially” or “substantially free/pure” with respect to a crystalline form means that the form contains about less than 30 percent, about less than 20 percent, about less than 15 percent, about less than 10 percent, about less than 5 percent, or about less than 1 percent by weight of impurities. Impurities may, for example, include other polymorphic forms, water, and solvents other than that in a designated polymorphic form.

As used herein and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more therapeutic agents to a patient with such a disease or disorder. In some embodiments, the terms refer to the administration of a compound provided herein, with or without other additional active agents, after the onset of symptoms of the particular disease. Entinostat has been studied in combination with hormone therapy for the treatment of breast cancer. Other studies have indicated that entinostat combined with immune checkpoint therapies may be most effective in “inflamed” tumors such as NSCLC and melanoma where immune checkpoint therapies have been previously tried but the disease nonetheless progressed.

As used herein and unless otherwise specified, the term “room temperature” refers to ambient temperature or the working laboratory temperature range, about 18° C. to about 25° C.

As used herein and unless otherwise specified, the term “atmospheric pressure” refers to about 760 mm Hg.

It is therefore an object of the present disclosure to provide Form A of entinostat compound with maleic acid, Form A of entinostat compound with succinic acid, Form B of entinostat compound with succinic acid, and Form C of entinostat compound with succinic acid that are substantially pure, stable, and scalable. It is also an object of the present disclosure to provide Form A of entinostat compound with maleic acid, Form A of entinostat compound with succinic acid, Form B of entinostat compound with succinic acid, and Form C of entinostat compound with succinic acid that are capable of being isolated and handled.

Techniques for characterizing crystal forms include but are not limited to differential scanning calorimetry (DSC), thermal gravimetric analysis (TGA), gravimetric vapor sorption (GVS), X-ray powder diffractometry (XRPD), and proton nuclear magnetic resonance (1H-NMR), and solubility assessments.

### XRPD

XRPD diffractograms are collected on a Bruker D8 diffractometer using Cu Ka radiation (40 kV, 40 mA) and a 0-20 goniometer fitted with a Ge monochromator. The incident beam passes through a 2.0 mm divergence slit followed by a 0.2 mm anti-scatter slit and knife edge. The diffracted beam passes through an 8.0 mm receiving slit with 2.5° Soller slits followed by the Lynxeye Detector. The software used for data collection and analysis is Diffrac Plus XRD Commander and Diffrac Plus EVA respectively. Samples are run under ambient conditions as flat plate specimens using powder as received. The sample is prepared on a polished, zero-background (510) silicon wafer by gently pressing onto the flat surface or packed into a cut cavity. The sample is rotated in its own plane.

The details of the standard data collection method are:
Angular range: 2 to 42° 20
Step size: 0.05° 20
Collection time: 0.5 s/step (total collection time: 6.40 min)

### ¹H NMR

¹H NMR spectra are collected on a Bruker 400 MHz instrument equipped with an auto-sampler and controlled by a DRX400 console or AV3+console with SMART probe. Samples are prepared in DMSO-d₆ solvent, unless otherwise stated. Automated experiments are acquired using ICON-NMR configuration within Topspin software, using standard Bruker-loaded experiments (¹H, ¹³C {1H}, DEPT135). Off-line analysis is performed using ACD Spectrus Processor.

### DSC

DSC data are collected on a TA Instruments Discovery DSC equipped with a 50 position auto-sampler. Typically, 0.5-3 mg of each sample, in a pin-holed aluminum pan, is heated at 10° C./min from 25° C. to 300° C. A purge of dry nitrogen at 50 ml/min is maintained over the sample. The instrument control software is TRIOS and the data are analysed using TRIOS or Universal Analysis.

### TGA

TGA data are collected on a TA Instruments Discovery TGA, equipped with a 25 position auto-sampler. Typically, 5-10 mg of each sample is loaded onto a pre-tared aluminum DSC pan and heated at 10° C./min from ambient temperature to 350° C. A nitrogen purge at 25 ml/min is maintained over the sample. The instrument control software is TRIOS and the data are analysed using TRIOS or Universal Analysis.

### GVS

Sorption isotherms are obtained using a Hiden IGASorp moisture sorption analyser, controlled by Isochema HISorp software. The sample temperature is maintained at 25° C. by a Grant LT ecocool 150 re-circulating water bath. The humidity is controlled by mixing streams of dry and wet nitrogen, with a total flow rate of 250 ml/min. The relative humidity is measured by a calibrated Vaisala RH probe (dynamic range of 0-95% RH), located near the sample. The weight change, (mass relaxation) of the sample as a function of % RH is constantly monitored by the microbalance (accuracy±0.001 mg). Typically, 20-30 mg of sample is placed in a tared mesh stainless steel basket under ambient conditions. The sample is loaded and unloaded at 40% RH and 25° C.(typical room conditions). A moisture sorption isotherm is performed as outlined below (2 scans, giving 1 complete cycle). The standard isotherm is performed at 25° C. at 10% RH intervals over a 0-90% RH range.

| Parameters | Values |
|---|---|
| Adsorption - Scan 1 | 40-90 |
| Desorption/Adsorption - Scan 2 | 90-0, 0-40 |
| Intervals (% RH) | 10 |
| Number of Scans | 2 |
| Flow rate (ml/min) | 250 |
| Temperature (° C.) | 25 |
| Stability (° C./min) | 0.05 |
| Minimum Sorption Time (mins) | 10 |
| Maximum Sorption Time (mins) | 360 |
| Equilibration Mode | Final Rate |
| Accuracy (tolerance) | +/−0.001 mg/min (over 600 s) |

### 4 Hour Solubility

Sufficient sample is suspended in 1.0 ml media for a maximum anticipated concentration of about 10 mg/ml of the free form of the compound. The resulting suspensions are then shaken at 25° C./750 rpm for 4 hours. The pH of the sample solutions is checked after 1 hour to ensure that the desired pH is maintained throughout (+0.2). After equilibration, the appearance is noted, and the final pH of the saturated solution is measured. Samples are then filtered through a glass ‘C’ fibre filter (Particle retention size 1.2 μm), before dilution with buffer as appropriate. Quantitation is by HPLC with reference to a standard solution of approximately 0.15 mg/ml. Different volumes of the standard, diluted and undiluted sample solutions are injected. The solubility is calculated using the peak areas determined by integration of the peak found at the same retention time as the principal peak in the standard injection.

The HPLC method for solubility measurements is as follows:

| Parameter | Value | | |
|---|---|---|---|
| Type of method | Reverse phase with gradient elution | | |
| Column | Phenomenex Luna, C18 (2) 5 μm 50 × 4.6 mm | | |
| Column Temperature | 25 | Autosampler | Ambient |
| (° C.) | | temperature (° C.) | |
| Standard Injections (μl) | 1, 2, 3, 4, 5, 7 | | |
| Sample Injections (μl) | 1-20 | | |
| Detection: Wavelength & | 260, 90 | | |
| Bandwidth (nm) | | | |
| Flow Rate (ml/min) | 2.0 | | |
| Mobile Phase A | 0.1% TFA in Water | | |
| Mobile Phase B | 0.085% TFA in Acetonitrile | | |
| | Time (min) | % Phase A | % Phase B |
| Timetable | 0.0 | 95.0 | 5.0 |
| | 1.0 | 80.0 | 20.0 |
| | 2.3 | 5.0 | 95.0 |
| | 3.3 | 5.0 | 95.0 |
| | 3.5 | 95.0 | 5.0 |
| | 4.4 | 95.0 | 5.0 |

Analysis is performed on an Agilent HP1100/Infinity II 1260 series system equipped with a diode array detector and using OpenLAB software.

### The Details of the Solubility Media are as Follows:

| | | Final |
|---|---|---|
| Media | Preparation | pH |
| pH 1.2 | 12.5 ml of a 0.2M KCl solution is mixed with 21.25 | 1.2 |
| Hydrochloric | ml 0.2M HCl in a 50 ml volumetric flask, and then | |
| Acid Buffer | made to volume with deionized water. | |
| pH 2.8 | 0.39 g of sodium dihydrogen phosphate is dissolved | 2.8 |
| Buffer | in deionized water in 50 ml volumetric flask and | |
| | made up to near volume with deionized water. pH is | |
| | adjusted using phosphoric acid. | |
| pH 4.5 | 0.3 g sodium acetate trihydrate is dissolved in | 4.5 |
| Acetate | 1.4 ml 2N acetic acid in a 100 ml volumetric flask, | |
| Buffer | and then made to volume with deionized water. | |
| pH 6.8 | 12.5 ml of a 0.2M monobasic potassium phosphate | 6.8 |
| Phosphate | solution is mixed with 5.6 ml 0.2M sodium | |
| Buffer | hydroxide in a 50 ml volumetric flask, and then | |
| | made to volume with deionized water. | |
| pH 7.5 | 12.5 ml of a 0.2M monobasic potassium phosphate | 7.5 |
| Phosphate | solution is mixed with 9.8 ml 0.2M sodium | |
| Buffer | hydroxide in a 50 ml volumetric flask, and then | |
| | made to volume with deionized water. pH is | |
| | adjusted using 0.2M NaOH. | |

In one embodiment, Form A of entinostat compound with maleic acid is prepared comprising:
a) dissolving a combination of entinostat and maleic acid in methanol to form a solution; and
b) cooling the solution to yield Form A of entinostat compound with maleic acid as a solid.

In one embodiment, the molar ratio of entinostat to maleic acid is about 1:0.5. In one embodiment, about 8-12 volumes (mL), particularly about 10 volumes, of methanol is used per weight (g) of entinostat. In one embodiment, the dissolving occurs at about 45-55° C., particularly at about 50° C. In one embodiment, the cooling is to about 0-10° C., particularly to about 5° C. In one embodiment, the cooling is at a rate of about 0.1° C./min. One embodiment further comprises filtering the isolated solid. A further embodiment comprises drying the isolated solid. Form A of entinostat compound with maleic acid can be dried by any conventional methods known to one of ordinary skill in the art. In one embodiment, it is dried under vacuum.

In another embodiment, Form A of entinostat compound with maleic acid is prepared comprising:
a) combining ethanol and a lyophilized solid comprising entinostat and maleic acid to form a slurry, wherein the molar ratio of entinostat to maleic acid is about 1:0.5; and
b) maturing the slurry to yield Form A of entinostat compound with maleic acid.

In one embodiment, the lyophilized solid comprising entinostat and maleic acid is amorphous. The volume of ethanol used is not critical as long as it is sufficient to form a slurry, rather than a volume sufficient to dissolve the lyophilized solid. In one embodiment, about 10 volumes (mL) of ethanol are used per weight (g) of entinostat in the lyophilized solid. In one embodiment, the maturing comprises temperature cycling at room temperature and at about 50° C., with about 4 hours at each temperature. In one embodiment, the maturing occurs for several days, for example, for about 1 to 4 days, particularly for about 4 days. In one embodiment, the maturing results in a suspension. In one embodiment, the suspension is filtered after the maturing step. In one embodiment, Form A of entinostat compound with maleic acid is dried. Form A of entinostat compound with maleic acid can be dried by any conventional methods known to one of ordinary skill in the art. In one embodiment, the drying is under vacuum.

In one embodiment, a lyophilized solid comprising entinostat and maleic acid is prepared comprising:
a) dissolving a mixture of entinostat and maleic acid in a solvent mixture of acetonitrile and water to form a solution, wherein the molar ratio of entinostat to maleic acid is about 1:0.5; and
b) freeze drying the solution to yield the lyophilized solid comprising entinostat and maleic acid.

In one embodiment, the lyophilized solid comprising entinostat and maleic acid is amorphous. In one embodiment, the ratio of acetonitrile to water in the solvent mixture is about 1:1. In one embodiment, about 25-35 volumes (mL), particularly about 30 volumes, of the solvent mixture is used per weight (g) of entinostat. In one embodiment, the dissolving further comprises sonicating the mixture of entinostat and maleic acid in the solvent mixture. In one embodiment, the freeze drying comprises freezing the solution in a dry ice/acetone bath. In a further embodiment, the freeze drying comprises use of a freeze dryer. In one embodiment, the freeze drying occurs over a few days, particularly, over about 2 days.

In another embodiment, Form A of entinostat compound with succinic acid is prepared comprising:
a) dissolving a combination of entinostat and succinic acid in methanol to form a solution; and
b) cooling the solution to yield Form A of entinostat compound with succinic acid as a solid.

In one embodiment, the molar ratio of entinostat to succinic acid is about 1:0.5-0.75. In one embodiment, the molar ratio of entinostat to succinic acid is about 1:0.5. In another embodiment, the molar ratio of entinostat to succinic acid is about 1:0.75. In one embodiment, about 8-12 volumes (mL), particularly about 10 volumes, of methanol are used per weight (g) of entinostat. In one embodiment, the dissolving occurs at about 45-55° C., particularly at about 50° C. In one embodiment, the cooling is to about 0-10° C., particularly to about 5° C. In one embodiment, the cooling is at a rate of about 0.1° C./min. One embodiment further comprises filtering the isolated solid. A further embodiment comprises drying the isolated solid. Form A of entinostat compound with succinic acid can be dried by any conventional methods known to one of ordinary skill in the art. In one embodiment, the drying is under vacuum.

In another embodiment, Form A of entinostat compound with succinic acid is prepared comprising:
a) combining a solvent selected from methanol and DMF and a lyophilized solid comprising entinostat and succinic acid to form a slurry, wherein the molar ratio of entinostat to succinic acid is about 1:0.5; and
b) maturing the slurry to yield Form A of entinostat compound with succinic acid.

In one embodiment, the lyophilized solid comprising entinostat and succinic acid is amorphous. In one embodiment, the solvent is methanol. In another embodiment, the solvent is DMF. The volume of solvent used is not critical as long as it is sufficient to form a slurry, rather than a volume sufficient to dissolve the lyophilized solid. In one embodiment, about 10 volumes of solvent are used per weight (g) of entinostat in the lyophilized solid. In one embodiment, the maturing comprises temperature cycling at room temperature and at about 50° C., with about 4 hours at each temperature. In one embodiment, the maturing occurs for several days, for example, for about 1 to 4 days, particularly for about 4 days. In one embodiment, the maturing results in a solution. In a further embodiment, the solution is subjected to evaporation to yield Form A of entinostat compound with succinic acid. Form A of entinostat compound with succinic acid can be dried by any conventional methods known to one of ordinary skill in the art. In one embodiment, the drying is under vacuum.

In one embodiment, the lyophilized solid comprising entinostat and succinic acid is prepared comprising:
a) dissolving a mixture of entinostat and succinic acid in a solvent mixture of acetonitrile and water to form a solution, wherein the molar ratio of entinostat to succinic acid is about 1:0.5; and
b) freeze drying the solution to yield the lyophilized solid comprising entinostat and succinic acid.

In one embodiment, the lyophilized solid comprising entinostat and succinic acid is amorphous. In one embodiment, the ratio of acetonitrile to water in the solvent mixture is about 1:1. In one embodiment, about 55-65 volumes (mL), particularly about 60 volumes, of the solvent mixture is used per weight (g) of entinostat. In one embodiment, the dissolving further comprises sonicating the mixture of entinostat and succinic acid in the solvent mixture. In one embodiment, the freeze drying comprises freezing the solution in a dry ice/acetone bath. In one embodiment, the freeze drying comprises use of a freeze dryer. In one embodiment, the freeze drying occurs over a few days, particularly, over about 3 days.

In another embodiment, Form B of entinostat compound with succinic acid is prepared comprising:
a) combining a solvent selected from ethyl acetate, isopropyl acetate, and methyl isobutyl ketone with a lyophilized solid comprising entinostat and succinic acid to form a slurry, wherein the molar ratio of entinostat to succinic acid is about 1:0.5; and
b) maturing the slurry to yield Form B of entinostat compound with succinic acid.

In one embodiment, the lyophilized solid comprising entinostat and succinic acid is amorphous. In one embodiment, the solvent is ethyl acetate. In another embodiment, the solvent is isopropyl acetate. In a further embodiment, the solvent is methyl isobutyl ketone. The volume of solvent used is not critical as long as it is sufficient to form a slurry, rather than a volume sufficient to dissolve the lyophilized solid. In one embodiment, about 10 volumes of solvent are used per weight (g) of entinostat in the lyophilized solid. In one embodiment, the maturing comprises temperature cycling at room temperature and at about 50° C., with about 4 hours at each temperature. In one embodiment, the maturing occurs for several days, for example, for about 1 to 4 days, particularly for about 4 days. In one embodiment, the maturing results in a suspension. In one embodiment, the suspension is filtered. Form B of entinostat compound with succinic acid can be dried by any conventional methods known to one of ordinary skill in the art. In one embodiment, the drying is under vacuum.

In one embodiment, the lyophilized solid comprising entinostat and succinic acid is prepared comprising:
a) dissolving a mixture of entinostat and succinic acid in a solvent mixture of acetonitrile and water to form a solution, wherein the molar ratio of entinostat to succinic acid is about 1:0.5; and
b) freeze drying the solution to yield the lyophilized solid comprising entinostat and succinic acid.

In one embodiment, the lyophilized solid comprising entinostat and succinic acid is amorphous. In one embodiment, the ratio of acetonitrile to water in the solvent mixture is about 1:1. In one embodiment, about 55-65 volumes (mL), particularly about 60 volumes, of the solvent mixture is used per weight (g) of entinostat. In one embodiment, the dissolving further comprises sonicating the mixture of entinostat and succinic acid in the solvent mixture. In one embodiment, the freeze drying comprises freezing the solution in a dry ice/acetone bath. In one embodiment, the freeze drying comprises use of a freeze dryer. In one embodiment, the freeze drying occurs over a few days, particularly, over about 3 days.

In another embodiment, Form C of entinostat compound with succinic acid is prepared comprising:
a) combining methyl ethyl ketone and a lyophilized solid comprising entinostat and succinic acid to form a slurry, wherein the molar ratio of entinostat to succinic acid is about 1:0.5; and
b) maturing the slurry to yield Form C of entinostat compound with succinic acid.

In one embodiment, the lyophilized solid comprising entinostat and succinic acid is amorphous. The volume of methyl ethyl ketone used is not critical as long as it is sufficient to form a slurry, rather than a volume sufficient to dissolve the lyophilized solid. In one embodiment, about 10 volumes of methyl ethyl ketone are used per weight (g) of entinostat in the lyophilized solid. In one embodiment, the maturing comprises temperature cycling at room temperature and at about 50° C., with about 4 hours at each temperature. In one embodiment, the maturing occurs for several days, for example, for about 1 to 4 days, particularly for about 4 days. In one embodiment, the maturing results in a paste, wherein the paste is evaporated to dryness. Form C of entinostat compound with succinic acid can be dried by any conventional methods known to one of ordinary skill in the art. In one embodiment, the drying is under vacuum.

In one embodiment, the lyophilized solid comprising entinostat and succinic acid is prepared comprising:
a) dissolving a mixture of entinostat and succinic acid in a solvent mixture of acetonitrile and water to form a solution, wherein the molar ratio of entinostat to succinic acid is about 1:0.5; and
b) freeze drying the solution to yield the lyophilized solid comprising entinostat and succinic acid.

In one embodiment, the lyophilized solid comprising entinostat and succinic acid is amorphous. In one embodiment, the ratio of acetonitrile to water in the solvent mixture is about 1:1. In one embodiment, about 55-65 volumes (mL), particularly about 60 volumes, of the solvent mixture is used per weight (g) of entinostat. In one embodiment, the dissolving further comprises sonicating the mixture of entinostat and succinic acid in the solvent mixture. In one embodiment, the freeze drying comprises freezing the solution in a dry ice/acetone bath. In one embodiment, the freeze drying comprises use of a freeze dryer. In one embodiment, the freeze drying occurs over a few days, particularly, over about 3 days.

An embodiment of the invention is directed to Form A of entinostat compound with maleic acid. Another embodiment of the invention is directed to Form A of entinostat compound with succinic acid. A further embodiment is directed to Form B of entinostat compound with succinic acid. An additional embodiment is directed to Form C of entinostat compound with succinic acid. A further embodiment of the invention is directed to any of these forms prepared by a process embodiment as described herein.

The present disclosure also encompasses a pharmaceutical composition comprising one or more of Form A of entinostat compound with maleic acid, Form A of entinostat compound with succinic acid, Form B of entinostat compound with succinic acid, and Form C of entinostat compound with succinic acid, and a pharmaceutically acceptable excipient. The pharmaceutical composition may be prepared according to any methods known in the art.

The present disclosure provides for use of one or more of Form A of entinostat compound with maleic acid, Form A of entinostat compound with succinic acid, Form B of entinostat compound with succinic acid, and Form C of entinostat compound with succinic acid in preparing a medicament for use in treating a disease.

The present disclosure provides for a method of treating disease by administering to a patient in need thereof a pharmaceutical composition comprising one or more of Form A of entinostat compound with maleic acid, Form A of entinostat compound with succinic acid, Form B of entinostat compound with succinic acid, and Form C of entinostat compound with succinic acid. Entinostat, including the co-crystals or salts described herein, may be used in combination with hormone therapy for the treatment of breast cancer and in combination with immune checkpoint therapies for the treatment of NSCLC and melanoma. Entinostat, including the co-crystals or salts described herein, may also be useful in treating malignant tumors including leukemia, colorectal cancer, ovarian cancer, oral cancer, lung carcinoma, breast carcinoma, prostate carcinoma, and melanoma and in treating autoimmune diseases. Entinostat, including the co-crystals or salts described herein, may also be used in preparing a medicament useful in treating any of the above-mentioned diseases.

The dosage of the pharmaceutical compositions may be varied over a wide range. Optimal dosages and dosage regimens to be administered may be readily determined by those skilled in the art, and will vary with the mode of administration, the strength of the preparation and the advancement of the disease condition. In addition, factors associated with the particular patient being treated, including patient's sex, age, weight, diet, physical activity, time of administration and concomitant diseases, will result in the need to adjust dosages and/or regimens.

### EXAMPLES

Examples 1-6 which follow herein provide exemplary embodiments of the various entinostat co-crystals or salts.

### Example 1—Preparation of Form a of Entinostat Compound with Maleic Acid

A solid mixture of entinostat (750 mg) and 0.5 eq maleic acid (120 mg) are dissolved in MeCN/water (1:1) (30 vol, 22.5 ml) with sonication. The solution is split into 4 ml vials with 1.5 ml in each (˜50 mg free form equivalent). The solutions are frozen in a dry ice/acetone bath and lyophilised over 2 days resulting in a co-amorphous material. The resulting co-amorphous material is wetted with ethanol (500 μl) and matured (RT/50° C.) for 4 days, with about 4 hour intervals at each temperature. After 4 days, the resulting gray suspension is filtered before drying under suction. Dry solids are analyzed by XRPD and determined to be Form A of entinostat compound with maleic acid.

Form A of entinostat compound with maleic acid is characterized by its XRPD pattern peaks. 2θ and relative % intensity values for peaks are shown in Table 1.

TABLE 1
Average Peak List for Form A of Entinostat
Compound with Maleic Acid diffractogram

| Angle | Intensity |
|---|---|
| (2-Theta °) | (%) |
| 10.7 | 3.1 |
| 13.2 | 4.1 |
| 16.2 | 4.3 |
| 18.1 | 30.5 |
| 18.6 | 100.0 |
| 19.5 | 7.0 |
| 19.8 | 3.6 |
| 20.5 | 2.9 |
| 20.7 | 2.2 |
| 21.2 | 2.7 |
| 22.4 | 6.3 |
| 22.9 | 2.0 |
| 23.2 | 2.2 |
| 24.0 | 2.5 |
| 25.2 | 6.8 |
| 25.6 | 2.3 |
| 26.0 | 15.7 |
| 26.6 | 4.1 |
| 27.4 | 4.1 |
| 27.9 | 3.8 |
| 28.1 | 4.7 |
| 28.6 | 3.5 |
| 29.2 | 5.6 |
| — | — |

The angle measurements are ±0.2° 2θ. In one embodiment, key defining peaks for Form A of entinostat compound with maleic acid include peaks at 18.6 and 18.1° 2θ. In another embodiment, key defining peaks for Form A of entinostat compound with maleic acid further comprise a peak at 26.0° 20.

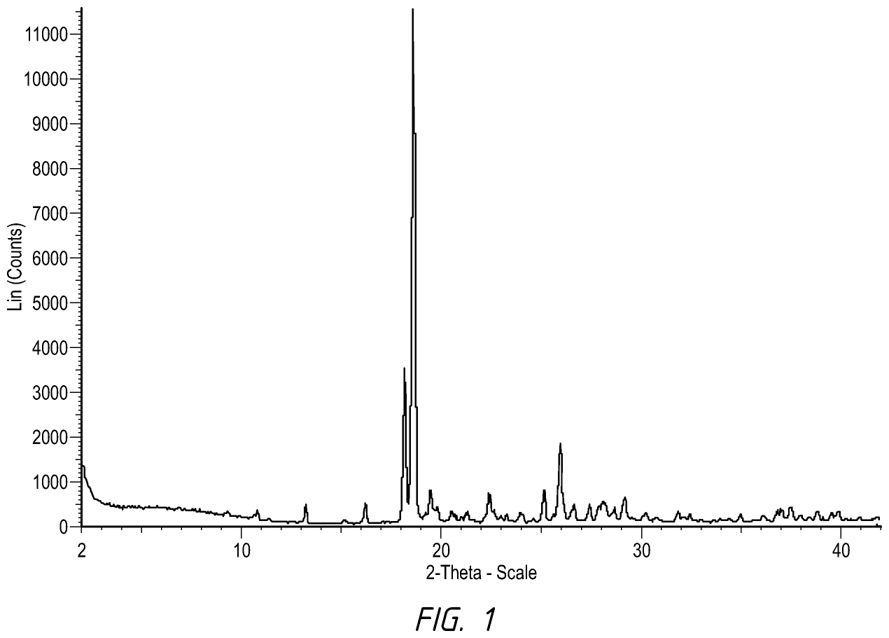

FIG. 1 provides a representative XRPD pattern of a representative sample of Form A of entinostat compound with maleic acid.

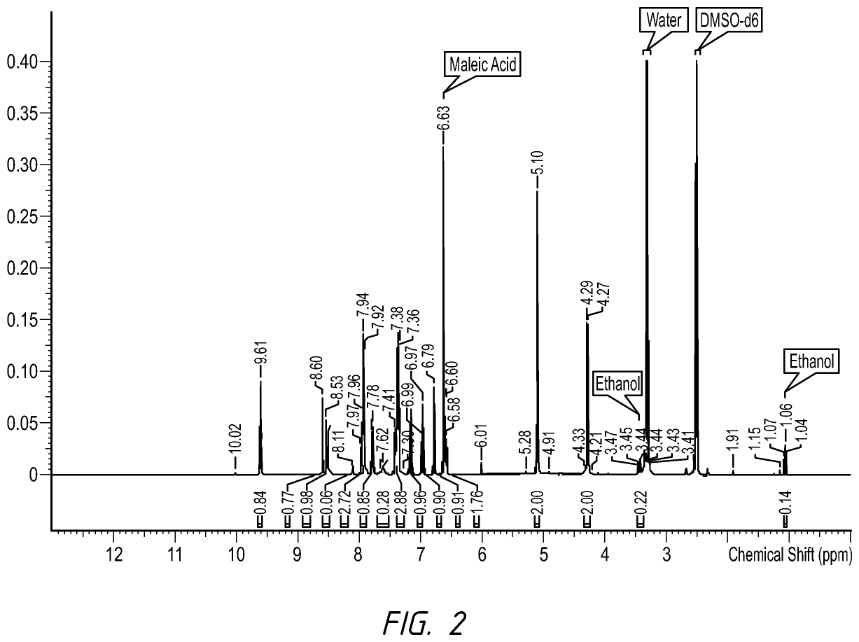

FIG. 2 provides a representative 1H NMR of Form A of entinostat compound with maleic acid which shows the presence of about 0.4 eq of maleic acid and trace amounts of residual ethanol.

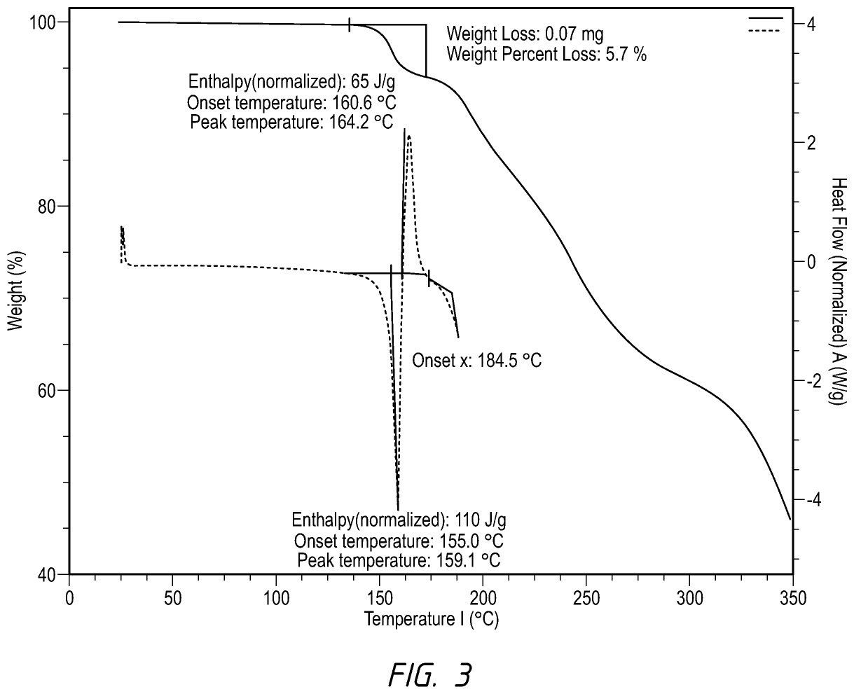

FIG. 3 provides a representative overlay of a TGA plot and DSC plot of Form A of entinostat compound with maleic acid. The TGA shows about a 5.7 wt % loss between about 135-170° C. and the DSC shows an endotherm with an onset at about 155° C., and an exotherm with an onset at about 161° C.

Form A of entinostat compound with maleic acid is stable at 40° C./75% RH for at least two weeks showing no change in form by XRPD.

### Example 2—Preparation of Form a of Entinostat Compound with Maleic Acid

A solid mixture of entinostat (50 mg) and 0.5 eq maleic acid (9.5 mg) is dissolved in methanol (500 μl, 10 vol) at 50° C. and cooled to 5° C. at 0.1° C./min. At 5° C., a suspension forms and is isolated by filtration before drying under suction. The solid is analyzed by XRPD and determined to be Form A of entinostat compound with maleic acid.

### Example 3—Preparation of Form a of Entinostat Compound with Succinic Acid

A solid mixture of entinostat (750 mg) and 0.5 eq succinic acid (122 mg) are dissolved in MeCN/water (1:1) (45 ml, 60 vol) with sonication. The solution is split into 4 ml vials with 3 ml in each (˜50 mg free form equivalent). The solutions are frozen in a dry ice/acetone bath and lyophilised over 3 days resulting in a co-amorphous material. The resulting co-amorphous material is wetted with methanol (500 μl) and matured (RT/50° C.) for 4 days, with about 4 hour intervals at each temperature. After 4 days, a clear solution is obtained and is left uncapped to evaporate the methanol, resulting in precipitation of solids. The solids are evaporated to dryness. Dry solids are analyzed by XRPD and determined to be Form A of entinostat compound with succinic acid.

Form A of entinostat compound with succinic acid is characterized by its XRPD pattern peaks. 20 and relative % intensity values for peaks are shown in Table 2.

TABLE 2
Average Peak List for Form A of Entinostat
Compound with Succinic Acid diffractogram

| Angle | Intensity |
|---|---|
| (2-Theta °) | (%) |
| 10.8 | 3.3 |
| 13.3 | 3.5 |
| 16.2 | 4.0 |
| 18.3 | 24.9 |
| 18.7 | 100.0 |
| 19.5 | 5.1 |
| 19.9 | 3.2 |
| 21.5 | 2.5 |
| 22.3 | 3.4 |
| 22.8 | 2.0 |
| 23.9 | 1.7 |
| 25.2 | 4.9 |
| 26.0 | 11.3 |
| 26.7 | 2.1 |
| 27.6 | 3.3 |
| 28.2 | 4.2 |
| 28.5 | 2.5 |
| 28.9 | 2.4 |
| 29.3 | 4.8 |

The angle measurements are ±0.2° 2θ. In one embodiment, key defining peaks for Form A of entinostat compound with succinic acid include peaks at 18.7 and 18.3° 2θ. In another embodiment, key defining peaks for Form A of entinostat compound with succinic acid further comprise a peak at 26.0° 2θ.

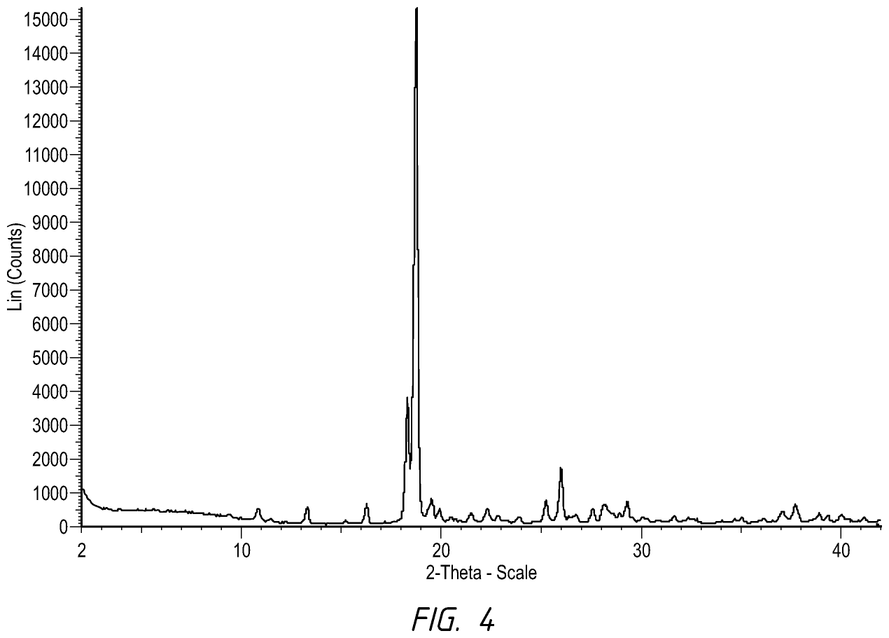

FIG. 4 provides a representative XRPD pattern of Form A of entinostat compound with succinic acid.

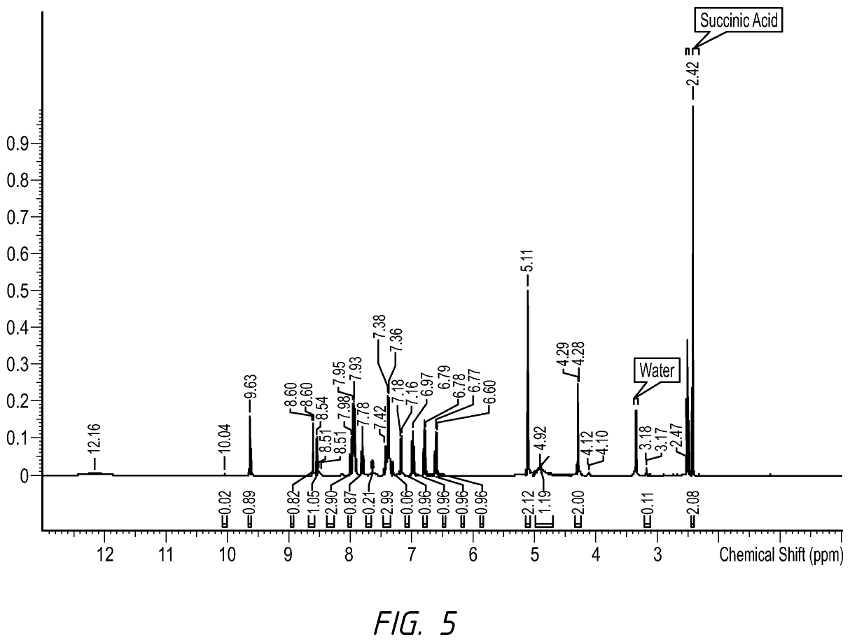

FIG. 5 provides a representative 1H NMR of Form A of entinostat compound with succinic acid which shows the presence of about 0.5 eq succinic acid and trace amounts of methanol.

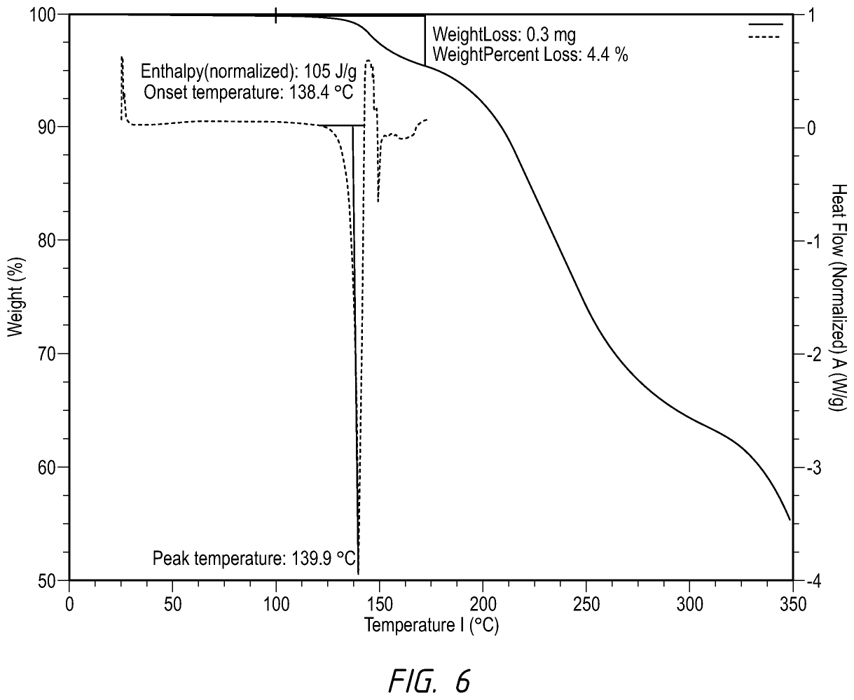

FIG. 6 provides a representative overlay of a TGA plot and DSC plot of Form A of entinostat compound with succinic acid. The TGA plot shows no weight loss until degradation. The DSC plot shows the onset of an endotherm at about 136° C.

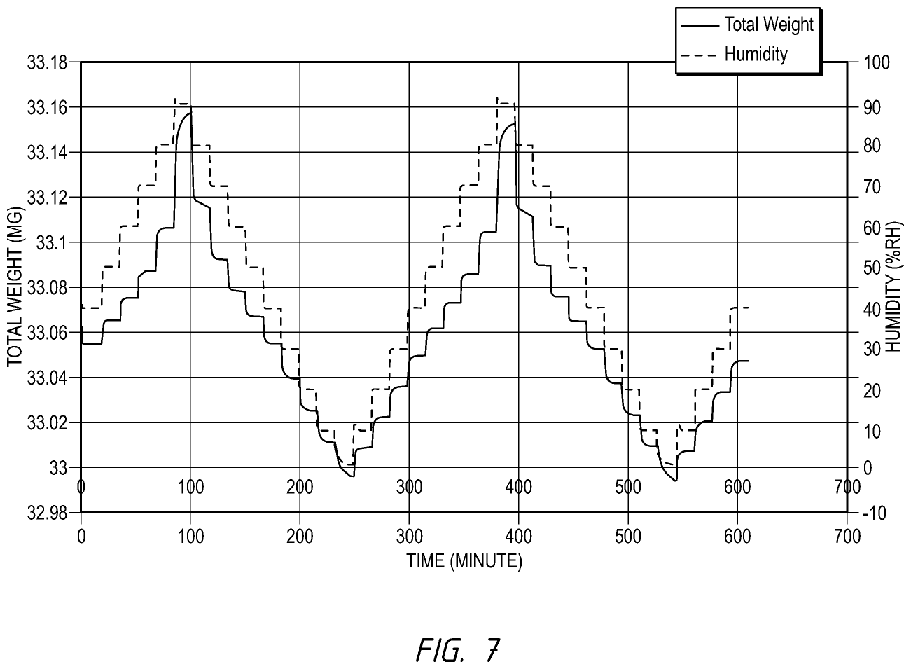

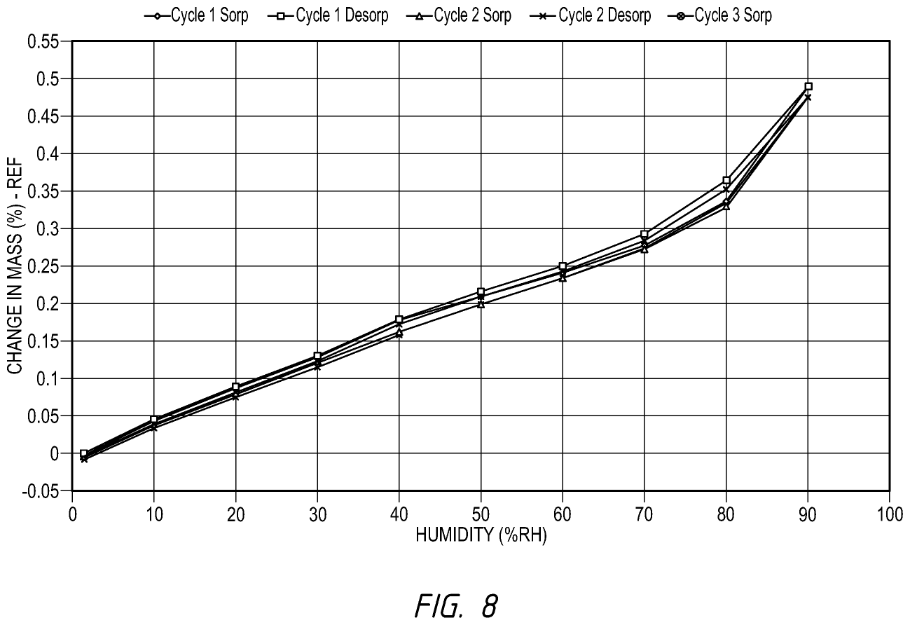

FIG. 7 provides a representative GVS kinetic plot of Form A of entinostat compound with succinic acid. FIG. 8 provides a representative GVS isotherm plot of Form A of entinostat compound with succinic acid. GVS shows about a 0.5 wt % reversible water uptake. Post GVS XRPD shows there is no change in crystalline form during the GVS.

Form A of entinostat compound with succinic acid remains stable at 40° C./75% RH and 25° C./97% RH for at least 28 days, as evidenced by XRPD.

The 4 hour solubility of Form A of entinostat compound with succinic acid compared to known forms of entinostat is provided below:

| | U.S. Patent | Form A of |
|---|---|---|
| U.S. Pat. | App. Pub No. | entinostat |
| No. 7,973,166 | 2020/0270213 | compound with |
| Polymorph A | Compound A | succinic acid |
| pH 1.2->7.9 mg/ml | pH 1.2->9.0 mg/ml | pH 1.2->9.0 mg/ml |
| pH 2.8-5.9 mg/ml | pH 2.8-1.9 mg/ml | pH 2.8-6.9 mg/ml |
| pH 4.5-0.19 mg/ml | pH 4.5-0.43 mg/ml | pH 4.5-0.86 mg/ml |
| pH 6.8-0.075 mg/ml | pH 6.8-0.34 mg/ml | pH 6.8-0.25 mg/ml |
| pH 7.5-0.072 mg/ml | pH 7.5-0.33 mg/ml | pH 7.5-0.22 mg/ml |

It is noted that the XRPD patterns for Form A of entinostat compound with maleic acid is similar to that of Form A of entinostat compound with succinic acid. The structures of maleic acid and succinic acid are very similar and it is possible that the entinostat compounds could have similar structures and therefore XRPD patterns.

Without wishing to be bound, it is believed that Form A of entinostat compound with succinic acid is an anhydrous hemi succinic acid co-crystal of entinostat.

### Example 4—Preparation of Form a of Entinostat Compound with Succinic Acid

Entinostat (500 mg) and succinic acid (0.5 eq, 84 mg) are dissolved in methanol (5 ml, 10 vol) at 50° C. The solution is then cooled to 5° C. at 0.1° C./min. After 2 days at 5° C., the resulting suspension is filtered and dried under suction. The isolated solids are dried under vacuum at RT for 1.5 hours. The solid is identified as Form A of entinostat compound with succinic acid by XRPD.

### Example 5—Preparation of Form B of Entinostat Compound with Succinic Acid

A solid mixture of entinostat (750 mg) and 0.5 eq succinic acid (122 mg) are dissolved in MeCN/water (1:1) (45 ml, 60 vol) with sonication. The solution is split into 4 ml vials with 3 ml in each (˜50 mg free form equivalent). The solutions are frozen in a dry ice/acetone bath and lyophilised over 3 days resulting in a co-amorphous material. The resulting co-amorphous material is wetted with methyl isobutyl ketone (500 μl) and matured (RT/50° C.) for 4 days, with about 4 hour intervals at each temperature. After 4 days, a suspension is obtained. The suspension is filtered and dried under suction. Dry solids are analyzed by XRPD and determined to be Form B of entinostat compound with succinic acid.

Form B of entinostat compound with succinic acid is characterized by its XRPD pattern peaks. 2θ and relative % intensity values for peaks are shown in Table 3.

TABLE 3
Average Peak List for Form B of Entinostat
Compound with Succinic Acid diffractogram

| Angle | Intensity |
|---|---|
| (2-Theta °) | (%) |
| 4.0 | 38.3 |
| 8.1 | 35.6 |
| 12.0 | 24.3 |
| 14.6 | 36.7 |
| 16.2 | 17.7 |
| 16.7 | 27.6 |
| 17.7 | 62.2 |
| 18.1 | 34.3 |
| 18.5 | 16.4 |
| 20.2 | 57.8 |
| 20.8 | 72.3 |
| 21.5 | 100.0 |
| 21.7 | 86.0 |
| 22.5 | 25.0 |
| 22.9 | 18.7 |
| 23.3 | 20.6 |
| 23.9 | 87.4 |
| 24.4 | 27.7 |
| 25.0 | 29.9 |
| 25.4 | 63.2 |
| 26.0 | 44.8 |
| 26.5 | 98.0 |
| 27.5 | 18.0 |
| 28.6 | 42.8 |
| 29.4 | 15.7 |
| — | — |

The angle measurements are ±0.2° 2θ. In one embodiment, key defining peaks for Form B of entinostat compound with succinic acid include at least two peaks selected from 21.5, 26.5, 23.9 and 21.7° 2θ. In another embodiment, key defining peaks for Form B of entinostat compound with succinic acid further comprise one or more peaks at 20.8, 25.4, and 17.7° 2θ.

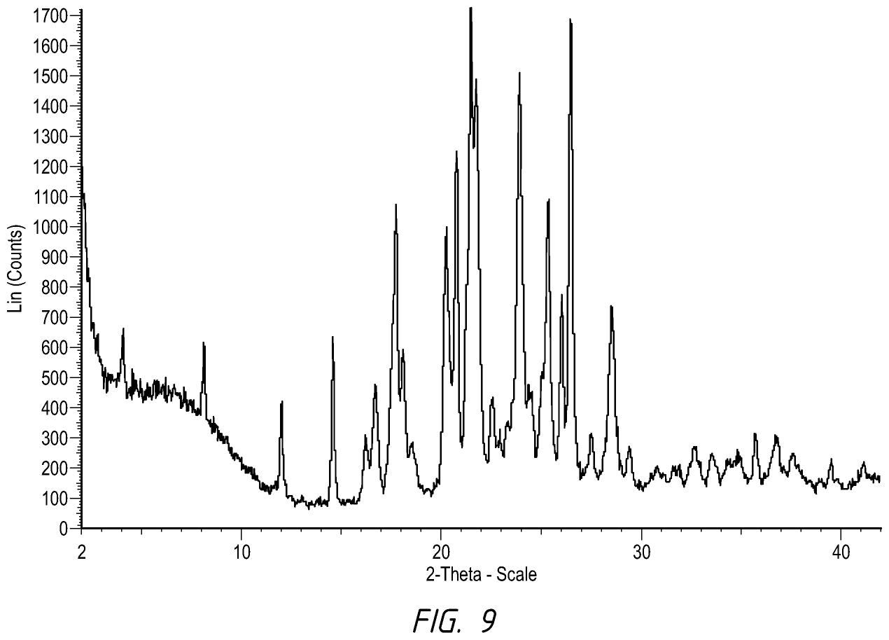

FIG. 9 provides a representative XRPD pattern of Form B of entinostat compound with succinic acid.

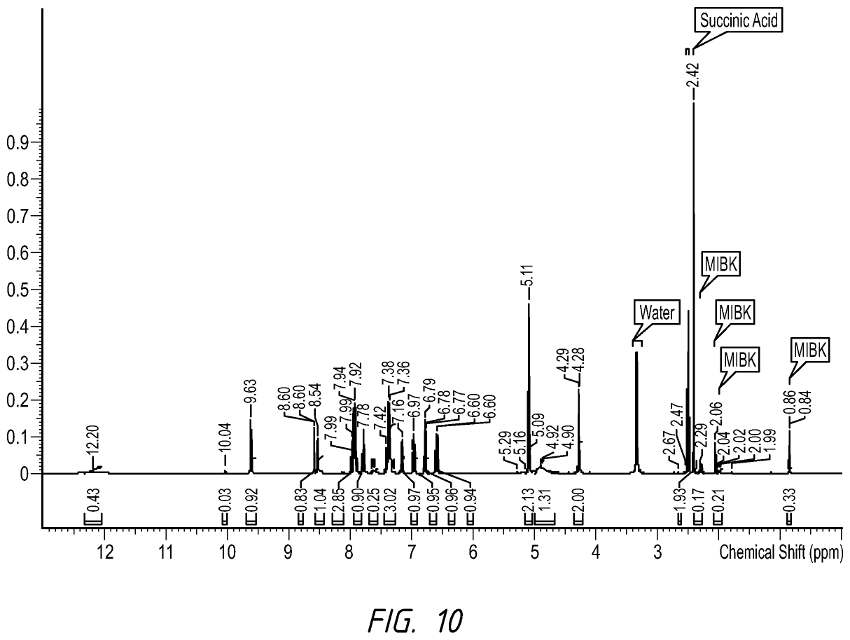

FIG. 10 provides a representative 1H NMR of Form B of entinostat compound with succinic acid which shows the presence of about 0.5 eq succinic acid and trace amounts of residual methyl isobutyl ketone.

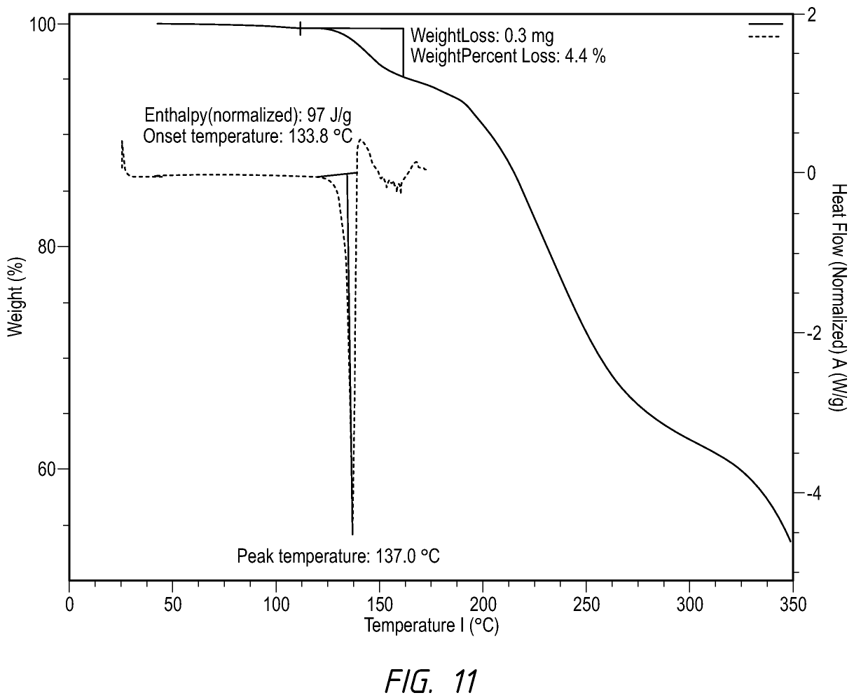

FIG. 11 provides a representative overlay of a TGA plot and DSC plot of Form B of entinostat compound with succinic acid. The TGA plot shows about a 4.4 wt % loss between bout 110-160° C. The DSC plot shows the onset of an endotherm at about 134° C.

Form B of entinostat compound with succinic acid remains stable at 40° C./75% RH for at least 7 days, as evidenced by XRPD.

### Example 6—Preparation of Form C of Entinostat Compound with Succinic Acid

A solid mixture of entinostat (750 mg) and 0.5 eq succinic acid (122 mg) are dissolved in MeCN/water (1:1) (45 ml, 60 vol) with sonication. The solution is split into 4 ml vials with 3 ml in each (˜50 mg free form equivalent). The solutions are frozen in a dry ice/acetone bath and lyophilised over 3 days resulting in a co-amorphous material. The resulting co-amorphous material is wetted with methyl ethyl ketone (500 μl) and matured (RT/50° C.) for 4 days, with about 4 hour intervals at each temperature. After 4 days, a paste is obtained. The paste is left uncapped to evaporate to dryness. Dry solids are analyzed by XRPD and determined to be Form C of entinostat compound with succinic acid.

Form C of entinostat compound with succinic acid is characterized by its XRPD pattern peaks. 2θ and relative % intensity values for peaks are shown in Table 4.

TABLE 4
Average Peak List for Form C of Entinostat
Compound with Succinic Acid diffractogram

| Angle | Intensity |
|---|---|
| (2-Theta °) | (%) |
| 4.0 | 100.0 |
| 8.0 | 91.2 |
| 12.8 | 26.0 |
| 15.7 | 65.5 |
| 18.5 | 22.6 |
| 19.0 | 60.0 |
| 19.4 | 41.5 |
| 19.9 | 12.1 |
| 20.7 | 28.5 |
| 21.2 | 7.4 |
| 22.0 | 12.3 |
| 22.3 | 11.1 |
| 22.6 | 17.3 |
| 24.1 | 34.3 |
| 24.8 | 12.6 |
| 25.0 | 17.6 |
| 25.7 | 9.6 |
| 26.0 | 24.9 |
| 26.7 | 37.3 |
| 27.7 | 23.2 |
| 28.1 | 14.9 |
| 29.4 | 13.1 |
| 29.7 | 9.7 |
| — | — |

The angle measurements are ±0.2° 2θ. In one embodiment, key defining peaks for Form C of entinostat compound with succinic acid include at least two peaks selected from 4.0, 8.0, 15.7, and 19.0° 2θ. In another embodiment, key defining peaks for Form C of entinostat compound with succinic acid further comprise one or more peaks at 19.4, 26.7, and 24.1° 2θ.

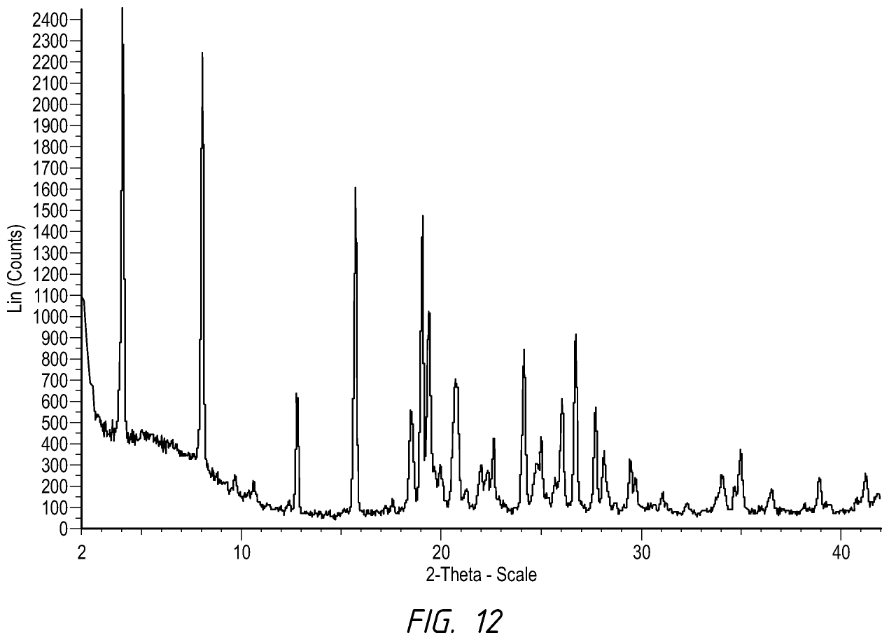

FIG. 12 provides a representative XRPD pattern of Form C of entinostat compound with succinic acid.

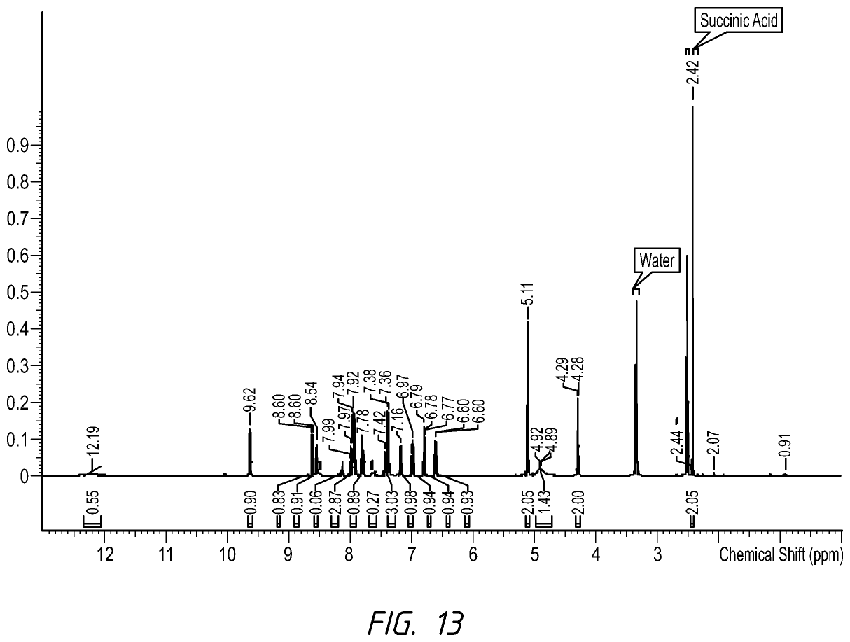

FIG. 13 provides a representative ¹H NMR of Form C of entinostat compound with succinic acid which shows the presence of about 0.5 eq succinic acid and no residual solvents present.

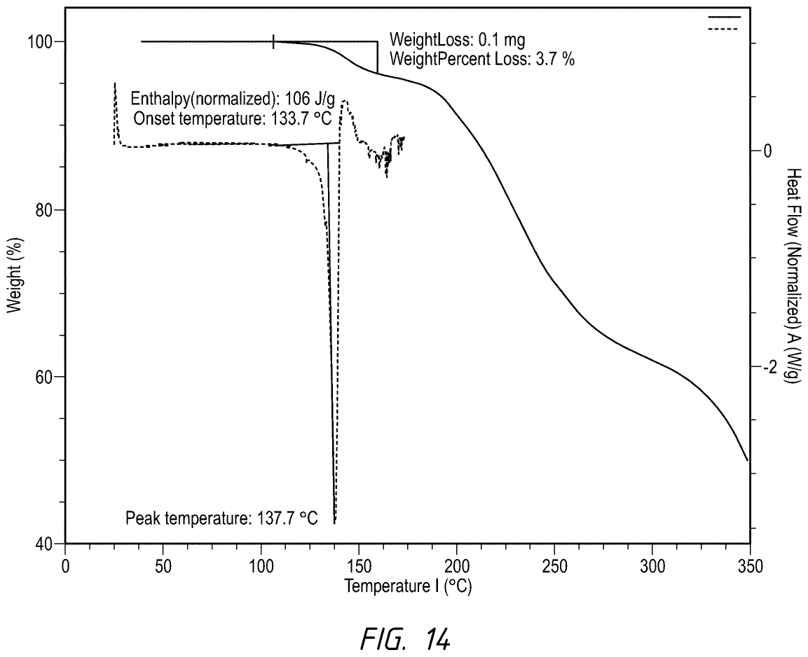

FIG. 14 provides a representative overlay of a TGA plot and DSC plot of Form C of entinostat compound with succinic acid. The TGA plot shows about a 3.7 wt % loss from about 105-160° C. The DSC plot shows the onset of an endotherm at about 134° C.

Form C of entinostat compound with succinic acid remains stable at 40° C./75% RH for at least 7 days, as evidenced by XRPD.

Without wishing to be bound, it is believed that Form A of entinostat compound with succinic acid, Form B of entinostat compound with succinic acid, and Form C of entinostat compound with succinic acid are polymorphs of a hemi succinic acid co-crystal of entinostat.

The above examples are set forth to aid in the understanding of the disclosure and are not intended and should not be construed to limit in any way the disclosure set forth in the claims which follow hereafter.

1.-11. (canceled)

12. Form A of entinostat with succinic acid, which is characterized by having X-ray powder diffraction peaks at about 26.0°, 18.7° and 18.3° 2θ±0.2° 2θ as measured by CuKa radiation.

13. Form A of entinostat with succinic acid according to claim 12, further characterized by onset of an endothermic event at about 136° C.±3° C., as measured by differential scanning calorimetry.

14. A method for preparing Form A of entinostat with succinic acid according to claim 12 comprising the steps of:
a) dissolving a combination of entinostat and succinic acid in methanol to form a solution; and
b) cooling the solution to yield Form A of entinostat with succinic acid as a solid.

15. The method of claim 14, wherein the molar ratio of entinostat to succinic acid is about 1:0.5-0.75.

16. A method for preparing Form A of entinostat with succinic acid according to claim 12 comprising the steps of:
a) combining a solvent selected from methanol and DMF with a lyophilized solid comprising entinostat and succinic acid to form a slurry, wherein the molar ratio of entinostat to succinic acid is about 1:0.5; and
b) temperature cycling the slurry from room temperature to about 50° C. to yield Form A of entinostat with succinic acid.

17. The method of claim 16, wherein the temperature cycling of the slurry is carried out at about 4 hours for each temperature.

18. The method of claim 16, wherein the temperature cycling of the slurry is carried out at about 4 days for each temperature.

19. Form B of entinostat with succinic acid, which is characterized by having 2 or more X-ray powder diffraction peaks selected from 17.7, 20.8, 21.5, 25.4, 26.5, 23.9 and 21.7° 2θ±0.2° 2θ as measured by CuKa radiation.

20. Form B of entinostat compound with succinic acid according to claim 19, which is further characterized by onset of an endothermic event at about 134° C.±3° C., as measured by differential scanning calorimetry.

21. A method for preparing Form B of entinostat compound with succinic acid according to claim 19 comprising:
a) combining a solvent selected from ethyl acetate, isopropyl acetate, and methyl isobutyl ketone with a lyophilized solid comprising entinostat and succinic acid to form a slurry, wherein the molar ratio of entinostat to succinic acid is about 1:0.5; and
b) temperature cycling the slurry from room temperature to about 50° C. to yield Form B of entinostat with succinic acid.

22. The method of claim 21, wherein temperature cycling of the slurry is carried out for about 4 hours at each temperature.

23. The method of claim 21, wherein temperature cycling of the slurry is carried out for about 4 days at each temperature.

24. Form C of entinostat with succinic acid, which is characterized by having 2 or more X-ray powder diffraction peaks selected from about 4.0, 8.0, 15.7, 19.0, 19.4, 26.7, and 24.1° 2θ±0.2° 2θ as measured by CuKa radiation.

25. Form C of entinostat with succinic acid according to claim 24, which is characterized by onset of an endothermic event at about 134° C.±3° C., as measured by differential scanning calorimetry.

26. A method for preparing Form C of entinostat compound with succinic acid according to claim 24 comprising:
a) combining methyl ethyl ketone and a lyophilized solid comprising entinostat and succinic acid to form a slurry, wherein the molar ratio of entinostat to succinic acid is about 1:0.5; and
b) temperature cycling the slurry from room temperature to about 50° C. slurry to yield Form C of entinostat with succinic acid.

27. The method of claim 26, wherein temperature cycling of the slurry is carried out for about 4 hours at each temperature.

28. The method of claim 26, wherein temperature cycling of the slurry is carried out for about 4 days at each temperature.

29. A pharmaceutical composition comprising a pharmaceutically effective amount of Form A of entinostat with succinic acid of claim 12.

30. A pharmaceutical composition comprising a pharmaceutically effective amount of Form B of entinostat with succinic acid of claim 19.

31. A pharmaceutical composition comprising a pharmaceutically effective amount of Form C of entinostat with succinic acid of claim 26 and a pharmaceutically acceptable excipient.
